(19) United States
(12) Patent Application Publication
(10) Pub. No.: US 2008/0214504 A1
(43) Pub. Date: Sep. 4, 2008

(54) Vitamin B6 Related Compounds and Methods for Recovery From Trauma

(75) Inventor: Albert Friesen, Winnipeg (CA)

Correspondence Address: MERCHANT & GOULD PC, P.O. BOX 2903, MINNEAPOLIS, MN, 55402-0903, US

(73) Assignee: MEDICURE INTERNATIONAL INC., St. James (BB)

(21) Appl. No.: 11/662,351

(22) PCT Filed: Sep. 12, 2005
(86) PCT No.: PCT/CA2005/001387

§ 371 Date: Feb. 19, 2008

Related U.S. Application Data
(60) Provisional application No. 60/608,409, filed on Sep. 10, 2004.

(51) Int. Cl. A61K 31/675; A61K 31/44; A61K 31/4355; A61P 43/00

(52) U.S. Cl. 514/89; 514/345; 514/302

(57) ABSTRACT

The present invention includes a method of promoting recovery from trauma in a patient in need thereof comprising administration of a therapeutically effective amount of a vitamin B6 related compound (excluding vitamin B6), preferable compounds include pyridoxal-5′-phosphate, 3-acylated analogues of pyridoxal, 3-acylated analogues of pyridoxy-4,5-animal, and pyridoxine phosphate analogues.

### FIELD OF INVENTION

The present invention relates to compositions useful for the treatment of a traumatic injury, and in particular surgical traumatic injury.

### BACKGROUND

Patients who suffer from an adverse physiological event such as stroke or myocardial infarction suffer traumatic injury resulting from damage to the vasculature. In such cases, the patient generally undergoes a surgical procedure to treat the damage resulting from the adverse event or to prevent further occurrences of the adverse event. Surgical intervention in and of itself is a form of further traumatic injury. It is desirable to improve functional recovery from such traumatic injury by either increasing the rate of recovery or by increasing the extent of recovery such that the functionality of the tissue and/or organ damaged as a result of the traumatic injury is close to or equal to its functionality prior to the traumatic injury. By improving tissue or organ function following a traumatic injury, or by increasing the rate of such recovery, or both, the affected patient's quality of life is greatly improved by reducing discomfort associated with repair of the damaged tissue and/or organ, reducing the number of days in spent in hospital and reducing the associated stress of undergoing treatment.

Currently, there is no safe and cost-effective pharmaceutical treatment, which reliably improves the prognosis of an individual who has suffered from a traumatic injury resulting from damage to the vasculature, and in particular a traumatic injury resulting from surgical intervention.

### SUMMARY OF INVENTION

The present invention provides a method of promoting patient recovery from trauma comprising administering a therapeutically effective amount of a vitamin B6 related compound.

In an embodiment of the invention, the vitamin B6 related compound is pyridoxal-5′-phosphate.

In a further embodiment of the invention, the vitamin B6 related compound is selected from a group comprising: pyridoxine, pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

In yet another embodiment of the invention, the trauma is caused by an adverse physiological event selected from a group consisting of: myocardial infarction, myocardial ischemia, ischemic stroke, hemorrhagic stroke, major cardiac trauma, hypertension, arteriosclerosis, aneurysm, and congestive heart failure.

In a still further embodiment of the invention, the trauma is the result of a traumatic contact suffered by the patient, the traumatic contact selected from a group consisting of: abrasion, incision, contusion, puncture, and compression.

In another embodiment of the invention, the trauma is a surgical trauma. The surgical trauma may be the result of a surgical procedure selected from a group consisting of: coronary bypass surgery, biopsy, heart valve replacement, atheroectomy, thrombectomy, transcatheter vascular therapy, angioplasty, vascular grafting, placement of a mechanical shunt, placement of an intravascular stent, and organ transplantation.

In a further embodiment of the invention, the vitamin B6 related compound is administered prior to, during, and/or following the surgical procedure.

### BRIEF DESCRIPTION OF THE FIGURES

Table 1 summarizes the baseline characteristics, length of hospitalization, and time to discharge from initiation of percutaneous coronary intervention (PCI) in patients treated with pyridoxal-5′-phosphate (P5P) and with placebo.

### DETAILED DESCRIPTION

The present invention is based upon the surprising discovery that functional recovery following a traumatic injury is significantly enhanced by the administration of pyridoxal-5′-phosphate, a vitamin B6 related compound.

The invention provides methods of treating a traumatic injury. In one aspect, the present invention provides a method of promoting recovery from trauma in a patient in need thereof, comprising administering a therapeutically effective amount of pyridoxal-5′-phosphate (P5P) or another vitamin B6 related compound.

As used herein, the term “promoting recovery” refers to the promotion of a clinically significant improvement to the physiological damage resulting from the traumatic injury.

While the exact mechanism for the trauma recovery promoting effects of vitamin B6 related compound is not known, the present inventors have found that administration of vitamin B6 related compounds increases the rate of recovery from trauma. Without being limited to one particular theory, the beneficial effects of vitamin B6 related compounds may be due in part to the compounds' abilities to promote tissue regeneration at the site of damage. The present inventors have previously shown the effectiveness of vitamin B6 related compounds for the treatment of various cardiovascular diseases (see for example U.S. Pat. Nos. 6,417,204 and 6,677,356). However, a surprising finding is that vitamin B6 related compounds are not only able to treat the adverse event caused by such diseases, they are also able to reduce or treat trauma.

As used herein, the term “vitamin B6 related compound” means any vitamin B6 related precursor, metabolite, derivative or analogue but excludes vitamin B6 (pyridoxine). In a preferred embodiment, the vitamin B6 related compound used to practice the invention is pyridoxal-5′-phosphate (P5P). Other vitamin B6 related compounds which can also be used to practice the invention, include the 3-acylated analogues of pyridoxal, 3-acylated analogues of pyridoxal-4,5-aminal, and pyridoxine phosphonate analogues described in U.S. Pat. No. 6,585,414 and US Patent Application No. 20030114424.

The 3-acylated analogues of pyridoxal include:

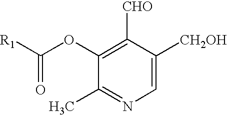

wherein,
R₁ is alkyl, alkenyl, in which alkyl can interrupted by nitrogen, oxygen, or sulfur, and can be unsubstituted or substituted at the terminal carbon with hydroxy, alkoxy, alkanoyloxy, alkoxyalkanoyl, alkoxycarbonyl, or
R₁ is dialkylcarbamoyloxy; alkoxy; dialkylamino; alkanoyloxy; alkanoyloxyaryl; alkoxyalkanoyl; alkoxycarbonyl; dialkylcarbamoyloxy; or
R₁ is aryl, aryloxy, arylthio, or aralkyl, in which aryl can be substituted by alkyl, alkoxy, amino, hydroxy, halo, nitro, or alkanoyloxy.

The 3-acylated analogues of pyridoxal-4,5-aminal include:

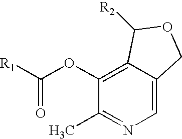

wherein,
R₁ is alkyl, alkenyl, in which alkyl can interrupted by nitrogen, oxygen, or sulfur, and can be unsubstituted or substituted at the terminal carbon with hydroxy, alkoxy, alkanoyloxy, alkoxyalkanoyl, alkoxycarbonyl, or
R₁ is dialkylcarbamoyloxy; alkoxy; dialkylamino; alkanoyloxy; alkanoyloxyaryl; alkoxyalkanoyl; alkoxycarbonyl; dialkylcarbamoyloxy; or
R₁ is aryl, aryloxy, arylthio, or aralkyl, in which aryl can be substituted by alkyl, alkoxy, amino, hydroxy, halo, nitro, or alkanoyloxy;
R₂ is a secondary amino group.

The pyridoxine phosphate analogues include:

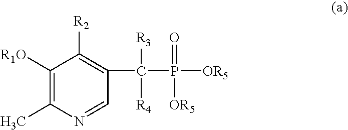

wherein,
R₁ is hydrogen or alkyl;
R₂ is —CHO—, —CH₂OH, —CH₃, —CO₂R₆ in which R₆ is hydrogen, alkyl, aryl; or
R₂ is —CH₂—O alkyl in which alkyl is covalently bonded to the oxygen at the 3-position instead of R₁;
R₃ is hydrogen and R₄ is hydroxy, halo, alkoxy, alkanoyloxy, alkylamino, or arylamino; or
R₃ and R₄ are halo; and
R₅ is hydrogen, alkyl, aryl, aralkyl, or —CO₂R₇ in which R₇ is hydrogen, alkyl, aryl, or aralkyl;

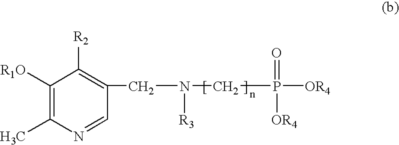

wherein,
R₁ is hydrogen or alkyl;
R₂ is —CHO, —CH₂OH, —CH₃, —CO₂R₅ in which R₅ is hydrogen, alkyl, aryl; or
R₂ is —CH₂—O alkyl in which alkyl is covalently bonded to the oxygen at the 3-position instead of R₁;
R₃ is hydrogen, alkyl, aryl, aralkyl,
R₄ is hydrogen, alkyl, aryl, aralkyl, or —CO₂R₆ in which R₆ is hydrogen, alkyl, aryl or aralkyl;
n is 1 to 6; and

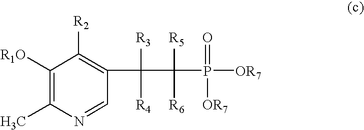

wherein,
R₁ is hydrogen or alkyl;
R₂ is —CHO—, CH₂OH—, —CH₃, —CO₂R₈ in which R₈ is hydrogen, alkyl, aryl; or
R₂ is —CH₂—O alkyl- in which alkyl is covalently bonded to the oxygen at the 3-position instead of R₁;
R₃ is hydrogen and R₄ is hydroxy, halo, alkoxy, or alkanoyloxy; or
R₃ and R₄ can be taken together to form ═O;
R₅ and R₆ are hydrogen; or
R₅ and R₆ are halo;
R₇ is hydrogen, alkyl, aryl, aralkyl, or —CO₂R₈ in which R₈ is hydrogen, alkyl, aryl, or aralkyl.

Some of the compounds described herein contain one or more asymmetric centres and this may give raise to enantiomers, diasteriomers, and other stereoisomeric forms which may be defined in terms of absolute stereochemistry as (R)- or (S)-. The present invention is meant to include all such possible diasteriomers and enantiomers as well as their racemic and optically pure forms. Optically active (R)- and (S)-isomers may be prepared using chiral synthons or chiral reagents, or resolved using conventional techniques. When the compounds described herein contain olefinic double bonds or other centres of geometric symmetry, and unless specified otherwise, it is intended that the compounds include both E and A geometric isomers. Likewise all tautomeric forms are intended to be included.

The invention is expected to be useful for treating a variety of traumatic injuries. The “traumatic injury” or “trauma” may be the result of tissue damage caused by an abrasion, incision, contusion, puncture, compression, etc. arising from traumatic contact with a foreign object such as during an accident or during a surgical procedure such as coronary bypass surgery, biopsy, heart valve replacement, atheroectomy, thrombectomy, transcatheter vascular therapy, angioplasty, vascular grafting, placement of a mechanical shunt, placement of an intravascular stent, or an organ transplantation. The “traumatic injury” or trauma may be the result of tissue damage caused by burns including chemical and radiation burns. The “traumatic injury” or “trauma” may be the result of tissue damage caused by an adverse physiological event such as myocardial infarction, myocardial ischemia, ischemic stroke, hemmorhagic stroke, major cardiac trauma, hypertension, arteriosclerosis, aneurysm, or congestive heart failure.

In practice, a patient suffering a traumatic injury is administered a therapeutically effective amount of vitamin B6 related compound shortly after the occurrence of the injury or the appearance of symptoms suggestive of a traumatic injury. In the case of “planned” traumatic injury, such as a scheduled surgical procedure, the patient may, in addition or in alternative, be administered a therapeutically effective amount of vitamin B6 related compound shortly before, or during, the planned trauma. The terms “effective amount” or a “therapeutically effective amount” of a compound refers to a nontoxic but sufficient amount of the compound to provide the desired effect. In the present invention, the “effective amount” of the vitamin B6 related compound is the amount that is effective to promote recovery from the traumatic injury. The amount that is effective will vary from subject to subject, and will depend on a number of factors which will be apparent to those skilled in the art and in light of the disclosure herein. In particular, these factors include: the identity of the compounds to be administered, the formulation, the route of administration employed, the patient's gender, age, and weight and the severity of the condition being treated and the presence of concurrent illness. Thus, it is not always possible to specify an exact “effective amount”. However, an appropriate effective amount in any individual case may be determined by one of ordinary skill in the art using routine experimentation. Methods for determining dosage and toxicity are well known in the art with studies generally beginning in animals and then in humans.

Where the vitamin B6 related compound employed is P5P, the typical therapeutic is between 1 to 1000 mg per kg patient per day. The preferred route of administration of the vitamin B6 related compound will depend on the particular traumatic injury to be treated and may include: oral administration, rectal administration, parental injection, and intravenous injection. It may be desirable to administer the vitamin B6 related compound with an appropriate pharmaceutically acceptable diluent or carrier known in the art.

In a preferred embodiment, the invention provides a method of promoting recovery from a surgical trauma in patient in need thereof, comprising the administration of a vitamin B6 related compound, and more preferably P5P. In a further preferred embodiment, the invention provides a method of promoting recovery from a surgical trauma resulting from surgical intervention to the cerebrovascular or cardiovascular system. The surgical trauma may include vascular trauma to the macrovasculature, microvasculature and/or the heart muscle.

Examples of surgical vascular traumas include, but are not limited to: (1) vascular surgery, such as coronary bypass surgery, biopsy, heart valve replacement, atheroectomy, thrombectomy, and the like; (2) transcatheter vascular therapies (TVT) including angioplasty, e.g., laser angioplasty and PTCA procedures employing balloon catheters, and indwelling catheters; (3) vascular grafting using natural or synthetic materials, such as in saphenous vein coronary bypass grafts, dacron and venous grafts used for peripheral arterial reconstruction, etc.; (4) placement of a mechanical shunt, such as a PIFE hemodialysis shunt used for arteriovenous communications; (5) placement of an intravascular stent, which may be metallic, plastic or a biodegradable polymer; and (6) organ transplantation, such as heart, kidney, liver and the like. Since the success of the post-operative recovery from surgical interventions such as the ones described herein is often a measure of the success of the surgery itself, treatment with B6 related compounds can improve the overall outcomes of such surgical procedures.

Individuals which maybe treated using the invention include those individuals about to undergo, undergoing or having undergone surgery. Administration of the vitamin B6 related compound may be initiated several days to weeks, prior to scheduled surgery and may be continued to and through the surgical procedure. Administration may also continue for several days to weeks or months post-surgery. For unscheduled surgery, the treatment may commence as soon as feasible prior to and throughout the surgical procedure.

In one embodiment, where the individual to be treated is undergoing vascular surgery, it is preferable to administer between 10 mg/kg patient weight and 30 mg/kg patient weight of P5P prior to the surgery. In a further preferred embodiment, the individual undergoing vascular surgery is administered 10 mg/kg of P5P orally at least 4 hours prior to the surgery and 10 mg/kg patient weight of P5P daily for at least 14 days following the surgery.

In may be desirable in certain circumstances to administer the vitamin B6 related compound in combination a cardioprotective agent following vascular surgery. Examples of cardioprotective agents which may be administered with the vitamin B6 related compound in order to promote trauma recovery include platelet aggregation inhibitor such as: thromboxane A₂ inhibitors (e.g. acetylsalicylic acid (ASA)), glycoprotein IIb/IIIa inhibitors (e.g. abciximab, eptifibatide, tirofiban, lamifiban, xemilofiban, orbofiban, sibrafiban, fradafiban, roxifiban, lotrafiban), adenosine phosphate inhibitors (e.g. clopidogrel, dipyridamole, sulfinpyrazone), fibrinogen-platelet binding inhibitors (ticlopidine), or a platelet c-AMP phosphodiesterase inhibitor, such as dipyridamole or cilostazol, or pentoxifylline (trental).

The vitamin B6 related compounds and the platelet aggregation inhibitors can be administered concurrently or successively following surgery. Therapeutically effective dosages for the platelet inhibitors discussed above are well known in the art. It may also be possible to slightly decrease the therapeutically effective dosage of platelet inhibitor (thus reducing the side effects of such treatment) as a result of the combination therapy with vitamin B6 related compound, due to the improved trauma recovery or speed of recovery that is a result of the vitamin B6 related compound treatment.

Although the invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLE ONE

### P5P Promotes Rapid Recovery Following PCI

Selection of Study Population—The patients selected for this trial were patients scheduled for elective percutaneous coronary intervention (PCI) with high-risk features for periprocedural myocardial infarction, and who satisfied specific inclusion criteria as described in the next section. Patients to be included in this study were of either sex, >18 years of age and admitted to hospital for elective, single-vessel, non-urgent PCI. Patients must have presented with at least one of the following high-risk clinical criteria:
ACS with last chest pain episode (or ischemic equivalent) within 48 hours,
recent myocardial infarction (≦7 days) with return of cardiac markers, (CK, CK-MB, and troponin) to below upper normal limits,
angiographic evidence of reduced epicardial flow (TIMI<3),
angiographic evidence of thrombus,
left ventricular ejection fraction≦30%, or
saphenous vein graft lesion.

The following patients were not eligible for inclusion into the study:
Those having suffered a recent myocardial infarction with elevated cardiac markers (CK-MB, troponin T), that have not returned to below upper limits or normal;
Those with electrocardiographic evidence of left bundle branch block (LBBB), ventricular paced rhythm, or atrial fibrillation;
Those with a planned multivessel PCI or PCI of known total occlusion (TIMI grade 0);
Those with evidence of ongoing or active clinical instability including; sustained systolic blood pressure<90 mmHg, cardiogenic shock, acute pulmonary edema or severe congestive heart failure, suspected acute myocarditis, pericarditis, endocarditis, cardiac tamponade, suspected dissecting aortic aneurysm, hemodynamically significant valvular heart disease, hypertrophic cardiomyopathy, restrictive cardiomyopathy, or congenital heart disease;
Any contraindication to the PCI procedure or any of the standard concomitant therapies used during routine PCI (e.g. aspirin, clopidogrel, heparin, low molecular weight heparin, direct antithrombin inhibitors, platelet glycoprotein IIb/IIIa antagonists);
Patients who were currently enrolled in a clinical trial of an investigational drug or who have participated in a clinical trial for an investigational drug (a new chemical entity not registered for clinical use) within 30 days preceding entry into the study or who are due to enter such a trial during the study period;
Patients with clinically significant abnormal laboratory findings (within 2 weeks prior to PCI) including: ALT, AST, bilirubin, or Alk Phos>1.5×ULN, Serum creatinine>1.8 mg/dL or 159 μmol/L;
Patients with any other pathology such as cancer, mental illness etc., which in the opinion of the investigator, might put the patient at risk or confound the results of the trial;
Female patients who are pregnant, breast feeding or planning a pregnancy during the course of the study; and
Patients who are unable or unwilling to comply with the protocol or who are not expected to complete the study period.

Treatments Administered—Following baseline laboratory assessments, patients were randomized to placebo or P5P (a vitamin B6 related compound), administered as a 10 mg/kg oral dose, at least (4) hours prior to PCI, followed by 10 mg/kg orally divided in two daily doses for fourteen (14) days. If PCI was delayed for more than 12 hours after initial dosing, they continued with scheduled BID dosing. If the initial loading dose was administered prior to or at 12:00 PM on day 1 of randomization, then the patient received a second evening dose. If the initial loading dose was administered following 12:00 PM, then no further dosing was administered on day 1, and the patient was to begin the next dose in the morning of day 2. If a dose was missed for more than 24 hours, patients were to continue to take the study drug as scheduled without taking supplemental doses. The date, time and dosing of the first and last study drug dosing was recorded in the CRF. Doses missed within a 24-hour period were to be amended by taking the scheduled dose for that day. Compliance was recorded as the number of tablets dispensed to the patient and returned to the site at the end of the study.

Selection and Timing of Dose for Each Patient—Following baseline laboratory assessments, patients were to be randomized to placebo or P5P administered as a 10 mg/kg oral dose given, at least (4) hours prior to PCI. This was followed by 10 mg/kg orally divided in two daily doses for fourteen (14) days. If the PCI was delayed for more than 12 hours after the initial dose, the patient continued with scheduled BID dosing. Also, if the initial loading dose was administered prior to or at 12:00 PM on day 1 of randomization, then the patient was to receive a second evening dose. If the initial loading dose was administered following 12:00 PM, then no further dosing was to be administered on day 1, and the patient should begin the next dose in the morning of day 2. If a dose was missed for more than 24 hours, patients continued to take the study drug as scheduled without taking supplemental doses. The date, time and dosing of the first and last study drug dosing was recorded in the CRF. Doses missed during a 24-hour period were amended, by taking the scheduled dose for that day. Compliance was recorded as the number of tablets dispensed to the patient and returned to the site at the end of the study.

Prior and Concomitant Therapy—At the time of PCI, all patients received 325 mg of aspirin and 300 mg of clopidogrel. Heparin was also administered in accordance with local standard of care. Thereafter, and for the next 30 days patients received 325 mg aspirin and 75 mg of clopidogrel daily. The choice of GP IIb/IIIa inhibition and additional medications was left to the discretion of the physician.

Results—Table 1 shows the baseline characteristics of patients participating in the study by treatment group. Patients randomized to P5P were treated 3 times more quickly (9.4 hours versus 31 hours), and were in hospital only half as long (3.3 days versus 7.0 days) as those given placebo. The number of days from initiation of the PCI procedure to discharge from the hospital was 74% shorter in patients treated with P5P versus those treated with placebo.

TABLE 1
Comparison of baseline characteristics, length of hospitalization and time
to discharge from initiation of PCI in patients treated with P5P
and placebo

| | P5P | Placebo | All Patients |
|---|---|---|---|
| | (N = 40) | (N = 20) | (N = 60) |
| Number of Diseased | | | |
| Vessels | | | |
| 0 | 1/40 (2.5%) | 0/20 | 1/60 (1.7%) |
| 1 | 19/40 (47.5%) | 14/20 (70.0%) | 33/60 (55.0%) |
| 2 | 13/40 (32.5%) | 2/20 (10.0%) | 15/60 (25.0%) |
| 3 | 5/40 (12.5% | 3/20 (15.0%) | 8/80 (13.3%) |
| Left Main | 2/40 (5.0%) | 1/20 (5.0%) | 3/60 (5.0%) |
| Primary reason for PCI | | | |
| ACS | 9/38 (23.7%) | 5/20 (25.0%) | 14/58 (24.1%) |
| Recent MI | 16/38 (42.1%) | 3/20 (15.0%) | 19/58 (32.8%) |
| Reduced eipcardial | 6/38 (15.8%) | 8/20 (40.0%) | 14/58 (24.1%) |
| flow | | | |
| Thrombus | 1/38 (2.6%) | 1/20 (5.0%) | 2/58 (3.5%) |
| LVEF <= 30 percent | 2/38 (5.3%) | 1/20 (5.0%) | 3/58 (5.2%) |
| Saphenous vein graft | 4/38 (10.5%) | 2/20 (10.0%) | 6/58 (10.3%) |
| lesion | | | |
| Length of | | | |
| hospitalization (days) | | | |
| N (Missing) | 40 (0) | 20 (0) | 60 (0) |
| Mean (S.D.) | 3.3 (5.5) | 7.0 (9.9) | 4.5 (7.4) |
| Median (25^{th}, 75^{th}) | 1.0 (1.0, 3.0) | 3.0 (1.0, 8.5) | 1.5 (1.0, 5.0) |
| Minimum, Maximum | 0.0, 34.0 | 1.0, 34.0 | 0.0, 34.0 |
| Time to discharge | | | |
| from initiation of | | | |
| PCI (days) | | | |
| N (Missing) | 37 (3) | 19 (1) | 56 (4) |
| Mean (S.D) | 1.9 (5.4) | 5.2 (10.3) | 3.0 (7.5) |
| Medium (25^{th}, 75^{th}) | 1.0 (1.0, 1.0) | 1.0 (1.0, 1.0) | 1.0 (1.0, 1.0) |
| Minimum, Maximum | 1.0, 34.0 | 1.0, 34.0 | 1.0, 34.0 |

1. A method of improving or promoting patient recovery from trauma comprising administering a therapeutically effective amount of a vitamin B6 related compound.

2. The method according to claim 1, wherein the trauma is caused by an adverse physiological event selected from the group consisting of: myocardial infarction, myocardial ischemia, ischemic stroke, hemorrhagic stroke, major cardiac trauma, hypertension, arteriosclerosis, aneurysm, and congestive heart failure.

3. The method according to claim 1, wherein the trauma is caused by a traumatic contact to the patient, said traumatic contact selected from the group consisting of:
abrasion, incision, contusion, puncture, compression, chemical burn, radiation burn, heat burn, and cold burn.

4. The method according to claim 1 wherein the trauma is surgical trauma resulting from the patient undergoing a surgical procedure.

5. The method according to claim 4, wherein the surgical procedure is selected from the group consisting of: coronary bypass surgery, biopsy, heart valve replacement, atheroectomy, thrombectomy, transcatheter vascular therapy, angioplasty, vascular grafting, placement of a mechanical shunt, placement of an intravascular stent, and organ transplantation.

6. The method according to claim 1, wherein the vitamin B6 related compound is selected from the group consisting of pyridoxine, pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

7. The method according to claim 1, wherein the vitamin B6 related compound is pyridoxal-5′-phosphate.

8. The method according to claim 6, wherein the 3-acylated analogue of pyridoxal is: 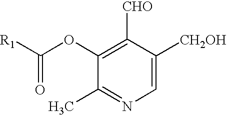
wherein,
R₁ is alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy,
alkoxy;
dialkylamino;
alkanoyloxy;
alkanoyloxyaryl;
alkoxyalkanoyl;
alkoxycarbonyl;
dialkylcarbamoyloxy;
aryl, in which aryl can be substituted by alkyl, alkoxy, amino, hydroxy, halo, nitro, or alkanoyloxy;
aryloxy;
arylthio; or
aralkyl; or
a pharmaceutically acceptable acid addition salt thereof.

9. The method according to claim 6, wherein the 3-acylated analogue of pyridoxal-4,5-aminal is 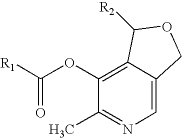
wherein,
R₁ is alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy;
aryl, in which aryl can be substituted by alkyl, alkoxy, amino, hydroxy, halo, nitro, or alkanoyloxy
aryloxy;
arylthi; or
aralkyl; and
R₇ is of the formula 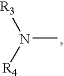
wherein R₃ and R₄ are each independently alkyl, alkenyl, cycloalkyl, aryl, or, when R₃ and R₄ are taken together to form a ring with the nitrogen atom, which may optionally be interrupted by a heteroatom; or
a pharmaceutically acceptable acid addition salt thereof.

10. The method according to claim 6, wherein the pyridoxine phosphate analogue is selected from a group consisting of: 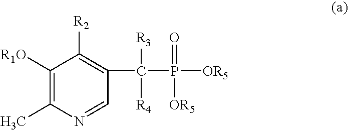
wherein,
R₁ is hydrogen or alkyl;
R₂ is —CHO—, —CH₂OH, —CH₃, —CO₂R₆ in which R₆ is hydrogen, alkyl, or aryl; or
R₂ is —CH₂—O alkyl in which alkyl is covalently bonded to the oxygen at the 3-position instead of R₁;
R₃ is hydrogen and R₄ is hydroxy, halo, alkoxy, alkanoyloxy, alkylamino, or arylamino; or
R₃ and R₄ are halo; and
R₅ is hydrogen, alkyl, aryl, aralkyl, or —CO₂R₇ in which R₇ is hydrogen, alkyl, aryl, or aralkyl; 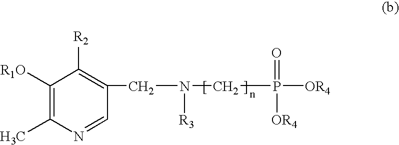
wherein,
R₁ is hydrogen or alkyl;
R₂ is —CHO, —CH₂OH, —CH₃, —CO₂R₅ in which R₅ is hydrogen, alkyl, or aryl; or
R₂ is —CH₂—O alkyl in which alkyl is covalently bonded to the oxygen at the 3-position instead of R₁;
R₃ is hydrogen, alkyl, aryl, or aralkyl,
R₄ is hydrogen, alkyl, aryl, aralkyl, or —CO₂R₆ in which R₆ is hydrogen, alkyl, aryl or aralkyl; and
n is 1 to 6; and 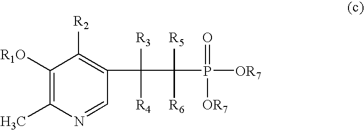
wherein,
R₁ is hydrogen or alkyl;
R₂ is —CHO—, CH₂OH—, —CH₃, —CO₂R₈ in which R₈ is hydrogen, alkyl, or aryl; or
R₂ is —CH₂—O alkyl- in which alkyl is covalently bonded to the oxygen at the 3-position instead of R₁;
R₃ is hydrogen and R₄ is hydroxy, halo, alkoxy, or alkanoyloxy; or
R₃ and R₄ can be taken together to form ═O;
R₅ and R₆ are hydrogen; or
R₅ and R₆ are halo;
R₇ is hydrogen, alkyl, aryl, aralkyl, or —CO₂R₈ in which R₈ is hydrogen, alkyl, aryl, or aralkyl.

11. The method according to claim 4, wherein the vitamin B6 related compound is administered prior to the patient undergoing the surgical procedure.

12. The method according to claim 11, wherein the vitamin B6 related compound is administered at least 4 hours prior to the patient undergoing the surgical procedure.

13. The method according to claim 11, further comprising administering a therapeutically effective amount of the vitamin B6 related compound following the surgical procedure.

14. The method according to claim 1, wherein the vitamin B6 related compound is administered after the trauma takes place.

15. The method according to claim 1, wherein the therapeutically effective amount of vitamin B6 related compound is between 1 and 1000 mg per day.

16. The method according to claim 7, wherein the therapeutically effective amount of pyridoxal-5′-phosphate is between 5 and 50 mg/kg patient weight per day.

17. The method according to claim 16, wherein the therapeutically effective amount of pyridoxal-5′-phosphate is 10 mg/kg patient weight per day.

18. The method according to claim 4, further comprising administering a platelet aggregation inhibitor selected from the group consisting of: a thromboxane A2 inhibitors, a glycoprotein IIb/IIIa inhibitor, an adenosine phosphate inhibitor, a fibrinogen-platelet binding inhibitor, and a platelet c-AMP phosphodiesterase inhibitor.

19. The method according to claim 18 wherein the administration of platelet aggregation inhibitor is at a lower dosage than a standard dose that would be given in the absence of vitamin B6 related compound administration.

20. A method of improving the success rate of surgical procedures comprising administering a therapeutically effective amount of a vitamin B6 related compound.

21. The method according to claim 20 wherein the surgical procedure is selected from the group consisting of coronary bypass surgery, biopsy, heart valve replacement, atheroectomy, thrombectomy, trans catheter vascular therapy, angioplasty, vascular grafting, placement of a mechanical shunt, placement of an intravascular stent, and organ transplantation.

22. The method according to claim 21, wherein the organ transplantation is selected from the group consisting of a kidney transplant, a heart transplant, and a liver transplant.

23. The method according to claim 20, wherein the vitamin B6 related compound is selected from the group consisting of pyridoxine, pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

24. The method according to claim 20, wherein the vitamin B6 related compound is pyridoxal 5 phosphate pyridoxal-5′-phosphate.

25. The method according to claim 23, wherein the 3-acylated analogue of pyridoxal is: 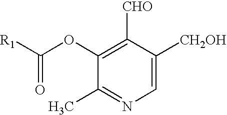
wherein,
R₁ is alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy;
aryl, in which aryl can be substituted by alkyl, alkoxy, amino, hydroxy, halo, nitro, or alkanoyloxy;
aryloxy;
arylthio; or
aralkyl; or
a pharmaceutically acceptable acid addition salt thereof.

26. The method according to claim 23, wherein the 3-acylated analogue of pyridoxal-4,5-aminal is 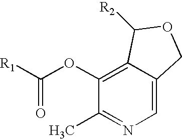
wherein,
R₁ is alkyl,
alkenyl,
in which alkyl or alkenyl
can be interrupted by nitrogen, oxygen, or sulfur, and
can be substituted at the terminal carbon by hydroxy,
alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy;
aryl, in which aryl can be substituted by alkyl, alkoxy, amino, hydroxy, halo, nitro, or alkanoyloxy
aryloxy;
arylthi; or
aralkyl; and
R₂ is of the formula 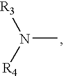
wherein R₃, and R₄ are each independently alkyl, alkenyl, cycloalkyl, aryl, or, when R₃ and R₄ are taken together to form a ring with the nitrogen atom, which may optionally be interrupted by a heteroatom; or
a pharmaceutically acceptable acid addition salt thereof.

27. The method according to claim 23, wherein the pyridoxine phosphate analogue is selected from the group consisting of: 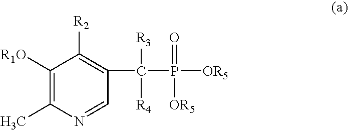
wherein,
R₁ is hydrogen or alkyl;
R₂ is —CHO—, —CH₂OH, —CH₃, —CO₂R₆ in which R₆ is hydrogen, alkyl, or aryl; or
R₂ is —CH₂—O alkyl in which alkyl is covalently bonded to the oxygen at the 3-position instead of R₁;
R₃ is hydrogen and R₄ is hydroxy, halo, alkoxy, alkanoyloxy, alkylamino, or arylamino; or
R₃ and R₄ are halo; and
R₅ is hydrogen, alkyl, aryl, aralkyl, or —CO₂R₇ in which R₇ is hydrogen, alkyl, aryl, or aralkyl; 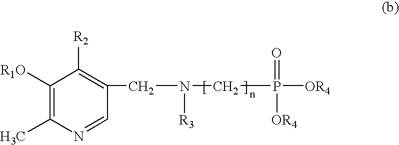
wherein,
R₁ is hydrogen or alkyl;
R₂ is 13 CHO, —CH₂OH, —CH₃, —CO₂R₅ in which R₅ is hydrogen, alkyl, or aryl; or
R₂ is —CH₂—O alkyl in which alkyl is covalently bonded to the oxygen at the 3-position instead of R₁;
R₃ is hydrogen, alkyl, aryl, aralkyl,
R₄ is hydrogen, alkyl, aryl, aralkyl, or —CO₂R₆ in which R₆ is hydrogen, alkyl, aryl or aralkyl; and
n is 1 to 6; and 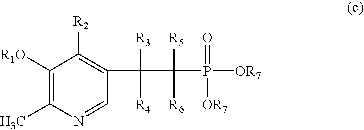
wherein,
R₁ is hydrogen or alkyl;
R₂ is —CHO—, CH₂OH—, —CH₃, —CO₂R₈ in which R₈ is hydrogen, alkyl, aryl; or
R₂ is —CH₂—O alkyl- in which alkyl is covalently bonded to the oxygen at the 3-position instead of R₁;
R₃ is hydrogen and R₄ is hydroxy, halo, alkoxy, or alkanoyloxy; or
R₃ and R₄ can be taken together to form ═O;
R₅ and R₆ are hydrogen or R₅ and R₆ are halo; and
R₇ is hydrogen, alkyl, aryl, aralkyl, or —CO₂R₈ in which R₈ is hydrogen, alkyl, aryl, or aralkyl.

28. The method according to claim 20, wherein the vitamin B6 related compound is administered prior to the patient undergoing the surgical procedure.

29. The method according to claim 28, wherein the vitamin B6 related compound is administered at least 4 hours prior to the patient undergoing the surgical procedure.

30. The method according to claim 28, wherein the step of administering a therapeutically effective amount of the vitamin B6 related compound following the surgical procedure.

31. The method according to claim 20, wherein the vitamin B6 related compound is administered after the trauma takes place.

32. The method according to claim 20, wherein the therapeutically effective amount of vitamin B6 related compound is between 1 and 1000 mg per day.

33. The method according to claim 24, wherein the therapeutically effective amount of pyridoxal 5′-phosphate is between 5 and 50 mg/kg patient weight per day.

34. The method according to claim 33, wherein the therapeutically effective amount of pyridoxal-5′-phosphate is 10 mg/kg patient weight per day.

35. The method according to claim 20, further comprising administering a platelet aggregation inhibitor selected from the group consisting of: a thromboxane A2 inhibitors, a glycoprotein IIb/IIIa inhibitor, an adenosine phosphate inhibitor, a fibrinogen-platelet binding inhibitor, and a platelet c-AMP phosphodiesterase inhibitor.

36. The method according to claim 35 wherein the administration of platelet aggreghan inhibitor is at a lower dosage than a standard dose that would be given in the absence of vitamin B6 related compound administration.
